(19) United States
(12) Patent Application Publication
(10) Pub. No.: US 2022/0240945 A1
(43) Pub. Date: Aug. 4, 2022

(54) EXTERNAL VASCULAR COMPRESSION DEVICE FOR USE DURING CARDIAC ARREST

(71) Applicant: Medical University of South Carolina, Charleston, SC (US)

(72) Inventors: Kristen Quinn, Mt Pleasant, SC (US); Heather Holman, Charleston, SC (US); Taufiek Konrad Rajab, Charleston, SC (US); Leah Plumblee, Johns Island, SC (US); Joshua Kim, Charleston, SC (US); Julie Siegel, Charleston, SC (US)

(21) Appl. No.: 17/591,178

(22) Filed: Feb. 2, 2022

Related U.S. Application Data
(60) Provisional application No. 63/144,599, filed on Feb. 2, 2021.

(51) Int. Cl. A61B 17/132; A61B 17/135; A61B 5/026; A61B 5/00

(52) U.S. Cl. CPC A61B 17/1325; A61B 17/1327; A61B 2017/00544; A61B 5/026; A61B 5/7405; A61B 17/135

(57) ABSTRACT

A vascular compression device is described. The device includes a first compression loop having a first pneumatic bulb configured to inflate and deflate, a second compression loop having a second pneumatic bulb configured to inflate and deflate, and a pneumatic pump system connected via tubing to the first and second pneumatic bulbs. A method for enhancing blood supply to vital organs is also described.

### CROSS-REFERENCE TO RELATED APPLICATIONS

This application claims priority to U.S. provisional application No. 63/144,599, filed Feb. 2, 2021, incorporated herein by reference in its entirety.

### BACKGROUND OF THE INVENTION

Despite decades of medical advancements, cardiopulmonary resuscitation (CPR) has remained largely unchanged since closed chest compressions were described in 1956. Morbidity and mortality remain dismal with only 10.6% of patients who experience cardiac arrest surviving until hospital discharge. Morbidity and mortality occur because of the limited blood supply to the vital organs such as the brain and heart. With closed chest compressions, blood supply is mechanically circulated and pharmacological manipulation of the vascular tree with drugs such as epinephrine and vasopressin are used to augment cardiac contractility and peripheral vasoconstriction. However, blood pressure (BP) can additionally be modulated by mechanical manipulation of the arterial tree (see e.g. Rajab and Schmitto, Medical Hypotheses Volume 83, Issue 1, July 2014, Pages 127-129). The systemic circulation has multiple parallel circuits that branch from the aorta. This anatomy permits a wide variation in regional blood flow to each of the parallel circulations at a given cardiac output (CO). Systemic vascular resistance (SVR) is related to the resistance in each of the parallel circuits (R1, R2 . . . Rn) according to the equation 1/SVR=1/R1+1/R2+1/Rn. Substituting this equation into Ohm's law yields BP=CO/(1/R1+1/R2+ . . . 1/Rn). Assuming a constant CO, removal of parallel circuits such as R2 from the circulation will thus increase BP.

Accordingly, what is needed is a device that can manipulate the vascular tree to increase blood supply to vascular beds supplying the vital organs during CPR and other medical emergencies that otherwise limit blood flow to these organs, such as shock and other states of hemodynamic instability.

### SUMMARY OF THE INVENTION

In one embodiment, a vascular compression device includes a first compression loop having a first pneumatic bulb configured to inflate and deflate, a second compression loop having a second pneumatic bulb configured to inflate and deflate, and a pneumatic pump system connected via tubing to the first and second pneumatic bulbs. In one embodiment, the first compression loop comprises a first strap and a first fastener, and the second compression loop comprises a second strap and a second fastener. In one embodiment, the pneumatic pump system comprises a manual pump. In one embodiment, the pneumatic pump system comprises an electric air pump. In one embodiment, the pneumatic pump system can be inflated with compressed air, O² or CO². In one embodiment, the device includes a pressure gauge connected to the tubing between the first and second pneumatic bulbs and the pneumatic pump system. In one embodiment, the tubing includes a first branch connected to the first pneumatic bulb and a second branch connected to the second pneumatic bulb, and wherein the first and second branch merge into a third branch connected to the pneumatic pump system. In one embodiment, the device includes a controller configured to communicate with the pneumatic system for inflating, holding inflation or deflating the first and second pneumatic bulbs. In one embodiment, at least one of the first and second pneumatic bulbs includes a pressure sensor communicatively connected to the controller. In one embodiment, at least one of the first and second pneumatic bulbs includes a physiological sensor communicatively connected to the controller. In one embodiment, the first pneumatic bulb has a first sensor communicatively connected to the controller and configured to detect flow occlusion, and the second pneumatic bulb has a second sensor communicatively connected to the controller and configured to detect flow occlusion. In one embodiment, the controller is configured to generate signals for inflating or deflating the first and second pneumatic bulbs based on feedback from the first and second pressure sensors. In one embodiment, the controller is configured to generate a sound cue based on feedback from the first and second pressure sensors. In one embodiment, at least one end of the of the firsts and second compression loops has a cuff slide portion comprising a firm low friction surface. In one embodiment, the tubing is configured to detach from the first and second pneumatic bulbs.

In one embodiment, a method for enhancing blood supply to vital organs includes the steps of applying localized external compression over an artery via an inflated bulb, and augmenting delivery of blood to increase blood pressure in the remaining circulation. In one embodiment, the step of applying localized compression further comprises applying compression over an artery supplying any or all extremities. In one embodiment, the step of applying localized compression further comprises applying compression comprises applying compression over two to four sets of arteries each supplying two to four separate extremities. In one embodiment, the step of applying localized compression further comprises applying compression over three or four sets of arteries each supplying three or four separate extremities. In one embodiment, the step of applying localized compression over an artery further comprises filling a bulb with air until a threshold pressure is detected and then maintaining that pressure. In one embodiment, the step of applying localized compression over an artery further comprises filling a bulb with air until a threshold physiological parameter is detected. In one embodiment, the step of applying localized compression over an artery further comprises filling a bulb with air until a threshold flow occlusion parameter is detected. In one embodiment the filling of the bulb maintains the occlusion parameter until manually disengaged. In one embodiment, the step of applying localized compression over an artery via an inflated bulb further comprises strapping the bulb to a surface patent's skin or over patient's clothing over the artery.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a perspective view of a vascular compression device according to one embodiment; FIG. 1B is an alternate perspective view of the device of FIG. 1A; and FIG. 1C is a top view of the device of FIG. 1A.

FIG. 2A is a diagram of a vascular compression device according to one embodiment; FIG. 2B is a top partially isolated view of a strap of FIG. 2A; and FIG. 2C is a side view of the strap of FIG. 2B.

FIG. 3 is an anatomical diagram illustration blood flow during localized compression according to one embodiment.

FIG. 4 is a top view of a vascular compression device according to one embodiment.

FIG. 5 is a chart of dimensional and material examples for a vascular compression device according to one embodiment.

FIG. 6 is a flow chart of a method for enhancing blood supply to vital organs.

### DETAILED DESCRIPTION OF THE INVENTION

It is to be understood that the figures and descriptions of the present invention have been simplified to illustrate elements that are relevant for comprehension of the present invention, while eliminating, for the purpose of clarity, many other elements found in vascular compression devices and methods of enhancing blood supply to vital organs. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. The description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a vascular compression device and method of enhancing blood supply to vital organs.

Embodiments of the device described herein are specifically designed to mechanically manipulate the vascular tree during CPR and other medical emergencies that normally limit blood flow to the vascular beds in vital organs. Embodiments of the device are implemented to occlude blood flow at the femoral artery and vein, bilaterally. Embodiments of the device can occlude blood flow at any extremity vessel to increase blood flow in the conserved, proximal vasculature. Embodiments of the vascular compression device can improve outcomes by boosting blood flow to vital organs such as the heart and brain during cardiac arrest or times of hemodynamic instability. The device is designed for rapid and intuitive deployment in wide array of settings, including but not limited to the inpatient hospital, clinical and rehabilitation settings, and in the field. Physiological sensors can be implemented to sense pulse pressure generated and read-out that information to the user. Both proximal (before device, towards body trunk) and distal (after device) pressures can be reported. The device should rapidly be wrapped around the supine (laying down) patient's legs, mechanically tightened, then bulbs inflated via the manual inflation pump.

One specific advantage over conventional devices is the pneumatic compression bulbs that deploy bilaterally or unilaterally with simple actuation, such as by the touch of a button or by rapid insufflation through compressed air. A pressure display and timer can be implemented to allow immediate and real-time feedback about the quality, force, and duration of extremity compression. These improvements offer a significant advantage over conventional devices used during treatment for cardiac arrest.

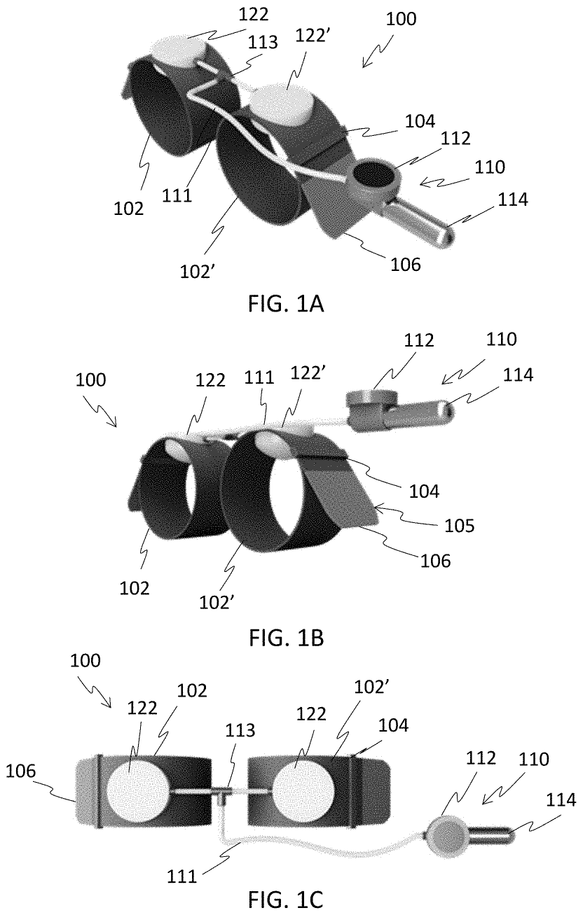

With reference now to FIGS. 1A-1C, a vascular compression device 100 according to one embodiment is shown. The vascular compression device 100 includes two compression loops 102, 102′ that in one embodiment can be formed as a strap and made from a dense but very flexible material that pulls tight around the extremity for which the device is applied. A fastener 104 such as a strap fastener secures the loop in place, and can further be reinforced by additional mechanisms such as a hook and loop portion 105. As will be apparent to those having ordinary skill in the art, other types of fasteners such as clasps, clamps, a self-locking ratchet system, a cinching system or other types of fasteners can be provided to implement the tourniquet feature. The compression loops are able to be separated and applied to any extremity. Each compression loop can include a bulb 122, 122′ connected by tubing 111 to a control section 110. The tubing 111 in certain embodiments can connect from each bulb 122, 122′ to a merged portion 113 then extend as a single tube to the control section 110. The control section 110 can include an air gauge 112 and a pump 114 for measuring and controlling pressure in each bulb. The pump 114 can be for example a manual pump or electric pump.

The control section 110 may also include a speaker for sound output, a microphone for audible input, and a communications unit for communicating with devices such as handheld mobile devices, electronic medical records, medical vital sign reporting devices, or laptops via wired or wireless communication. In one embodiment the control section is reusable and the two compression loops can be disconnected for cleaning or replacement. The central console can include several input elements such as buttons to deploy and release the pneumatic bulbs, and to navigate settings such as on an integrated display. The display can show the compression pressure achieved and an indication of whether extremity occlusion is achieved and to what degree. The display can display and record time as it passes, and the real-time blood pressures generated during an event. Pressure levels can be relayed in real-time to medical professionals treating the patient via display and/or audio feedback. Audio feedback can also be set to metronome feature which can be calibrated to match the beats for minute indicated in high-quality CPR performance. Feedback in certain embodiments include physiological feedback to monitor patient condition via physiological sensors, such as one or more sensors configured in the strap or bulb. In one embodiment, the control section qualifies CPR compressions as adequate or not, and can display the CPR rate while tracking if it's sufficient. Adequate or inadequate pressure levels and/or CPR rates can be notified by a series of alerts provided by the display and/or speaker. The pneumatic bulbs 122, 122′ can be filled by air, or alternatively filled by other techniques known in the art such as utilizing a gas or fluid. In one embodiment the central console contains a GPS tracker and can communicate with emergency personnel.

In one embodiment, soft materials such as the cuff materials can be made from a rip-stop material (such as rip-stop nylon) to protect from abrasion/damage during patient transportation and cuff application. Strapping/securing for the cuff should be secure enough to hold the cuffs together at occlusion pressure, even when the bulbs are inflated. The ends of the straps should transition into the cuff slide 106. The cuff slide 106 can be operated like a shoehorn to facilitate sliding the leg cuff straps under the patient's legs. The bulbs 122, 122′ can be made of a flexible yet durable rubber or neoprene that will not burst when inflated at a high pressure and is puncture resistant. The bulbs 122, 122′ can have internal ribs to maintain a bulb shape and integrity when inflated. The pressure gauge housing can be made of polypropylene.

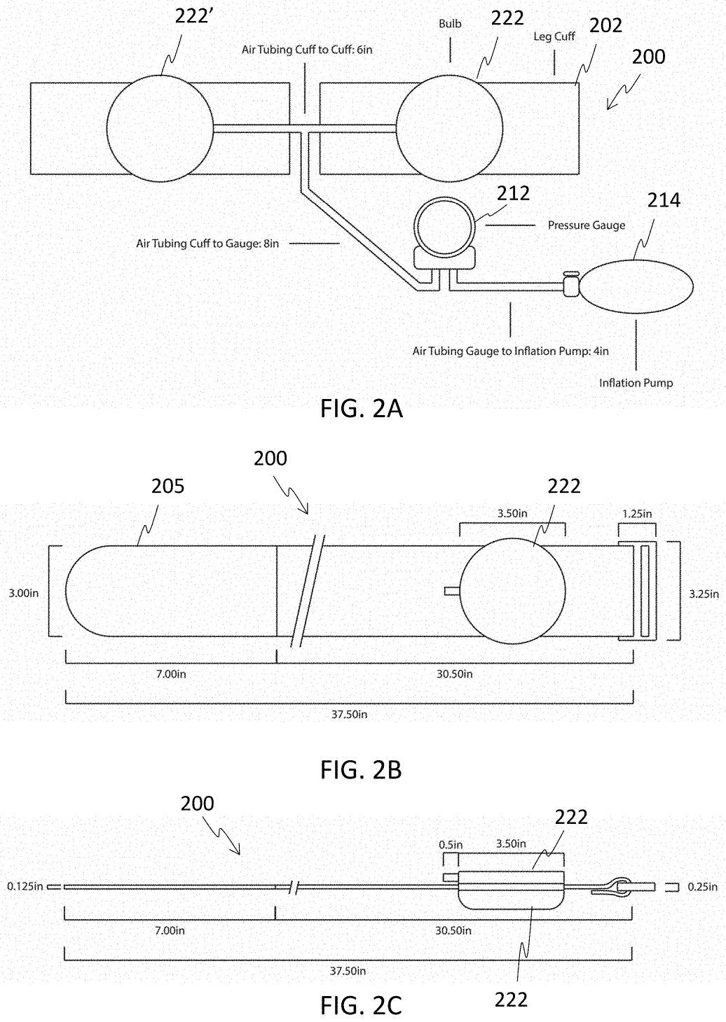

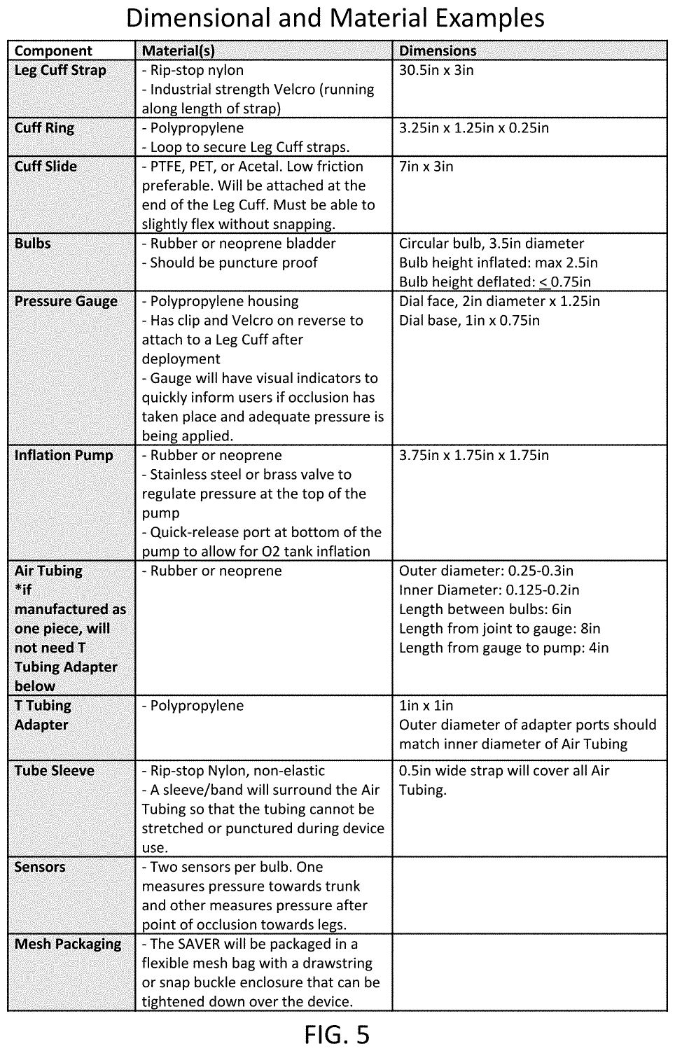

Various materials known in the art can be utilized to make components of the device to maximize functionality, durability, patient comfort, usability and deployment. It will be apparent to those having ordinary skill in the art that different sizes of the device can be provided, for example based on the age, height and weight of the person requiring treatment. With reference now to FIGS. 2A-2C, embodiments of the device 200 will be discussed to include exemplary sizing and material examples. In one embodiment, the leg cuff strap 202 includes at least one of a rip-stop nylon and an industrial strength Velcro running along the length of the strap. The strap can for example can be 20-50 inches long and 1.5-5 inches wide. In one embodiment the strap is substantially 30.5 inches×3 inches in dimension, with a thickness of 0.25 inches. In one embodiment, the cuff ring includes a polypropylene material. In one embodiment, the cuff slide 205 includes a PTFE, PET, or Acetal material. Low friction is preferable. The cuff slide will be attached at the end of the leg cuff and should be able to flex slightly without snapping. In one embodiment, the bulbs 222, 222′ include a rubber or neoprene bladder, and are preferably puncture proof. In one embodiment, the bulbs have substantially a 3.5 inch diameter, a 2.5 inch inflated height and a deflated height of less than 2.5 inches. In one embodiment, the pressure gauge 212 has a polypropylene housing. In one embodiment, the pressure gauge has clip or Velcro on a surface to attach to a leg cuff after deployment. In one embodiment, the pressure gauge has visual indicators to quickly inform users if occlusion has taken place and whether adequate pressure is being applied. In one embodiment, the inflation pump 214 is made from a rubber or neoprene material. In one embodiment, a stainless steel or brass valve is implemented to regulate pressure at the top of the pump. A quick-release port can be disposed at the bottom of the pump to allow for 02 tank inflation. In one embodiment, air tubing can be made from rubber or neoprene and can merge at a T tubing adapter, or if manufactures as an integral piece will not require a separate adapter. In one embodiment, the air tubing outer diameter ranges between 0.25 and 0.3 inches, and the inner diameter ranges between 0.125 and 0.2 inches. In one embodiment, the tubing length between bulbs is 6 inches, the length from joint to gauge is 8 inches, and the length from gauge to pump is 4 inches. The outer diameter of adapter ports can be sized to match the inner diameter of air tubing. A sleeve or band can surround the tubing so that the tubing cannot be stretched or punctured during device use. In one embodiment, a strap such as a 0.5 in wide strap will cover all air tubing. In one embodiment, two sensors per bulb are integrated into the device. One sensor can measure pressure towards trunk, and the other sensor can measure pressure after the point of occlusion towards the legs. Additional dimensional and material configuration examples are discussed in the chart of FIG. 5.

Accordingly, in certain embodiments, the device can be used for example outside of the setting of cardiopulmonary resuscitation to increase coronary and cerebral perfusion, in controlled settings such as the trauma bay and intraoperatively. The device can also be stationed at public venues or supplied to first responders that may have to administer CPR. In a typical scenario, the devices may be used when a patient experiences cardiac arrest and is unconscious. The medical professional positions the patient on their back and begins CPR. Ideally, a second person would assist and put the device's compression loops around each leg and pull tight to attach and fasten the strap to itself. The bulbs are soft and able to be easily adjusted over the femoral vasculature. A diagram on device packaging can ensure correct placement. Instructions can also be incorporated onto the display (including animated and audible instructions). The device will typically take less than one minute to get in place. A button on the display console can activate a pneumatic system that fills each bulb with air to occlude arterial circulation. These bulbs detect pressure which will be displayed on the central display. At the same time, a timer can start and display the total minutes and seconds the device has been deployed. This step confirms successful occlusion has been achieved. CPR should continue throughout this process. The device can be removed when return of spontaneous circulation is achieved.

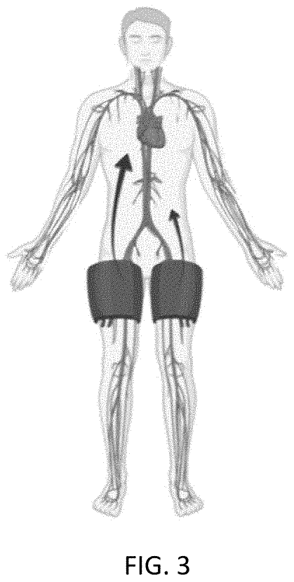

Advantageously, implementation is simple and tourniquets in the field take 60-90 seconds to deploy by an untrained individual. With reference to FIG. 3, 20% of a patient's blood volume can be contained in the lower extremities. That blood can be rerouted back to vital organs with embodiments of the device described herein. Tourniquets can be applied to lower extremities for up to 2 hours before damage occurs. In addition to CPR, the device is useful to compensate sudden changes in blood pressure, for example in surgery with high risk for hemodynamic instability or bleeding (hemorrhagic shock). In this case, the device is positioned and prepared ahead of time and activated by the medical team when necessary.

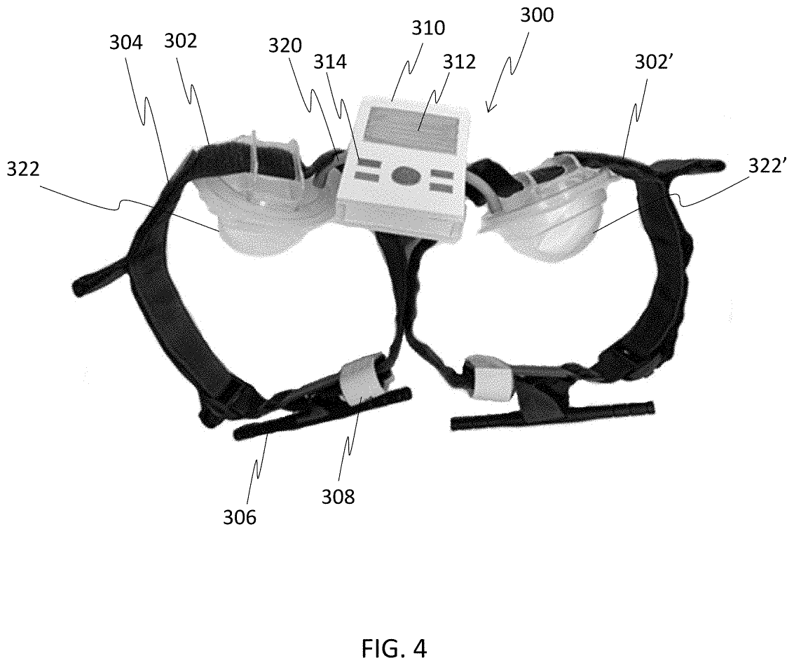

With reference now to FIGS. 4A-4C, a vascular compression device 300 according to one embodiment is shown. The vascular compression device 300 includes two compression loops 302, 302′ that in one embodiment can be made from a dense but very flexible material formed as a strap that pulls tight around the extremity for which the device is applied. A hook and loop fastener 304 such as Velcro secures the loop in place. As will be apparent to those having ordinary skill in the art, other types of fasteners such as clasps, a self-locking ratchet system, a cinching mechanism or other types of fasteners can be provided to implement the tourniquet feature. The compression loops are able to be separated and applied to any extremity. In one embodiment, they can be further tightened by twisting a windlass 306, which after twisting can be secured to the compression loop by an attachment 308. Each compression loop can include a plug-in connection to the central console 310 that houses a controller, a display 312 and buttons 314 for providing user input. The console can also house a speaker for sound output, a microphone for audible input, and a communications unit for communicating with devices such as handheld mobile devices or laptops via wired or wireless communication. In one embodiment the central console is reusable and the two compression loops can be disconnected for cleaning or replacement. The central console has several buttons to deploy and release the pneumatic bulbs, and to navigate through the display. The display shows the compression pressure achieved and an indication of whether extremity occlusion is achieved. Pressure is relayed in real-time to medical professionals treating the patient. In one embodiment the central console qualifies CPR compressions as adequate or not. In another embodiment the central console can display the CPR rate and determine adequacy or not. Adequate or inadequate pressure levels and/or CPR rates can be notified by a series of alerts provided by the display and/or speaker. The pneumatic bulbs 322, 322′ are filled by a pneumatic system 320 communicating with the controller 310, however in some embodiments there is an attached hand-pump to allow manual filling of the pneumatic bulbs. The controller can also communicate with other physiological sensors to provide real-time feedback and pressure adjustments. In another embodiment the central console contains a GPS tracker and can communicate with EMS.

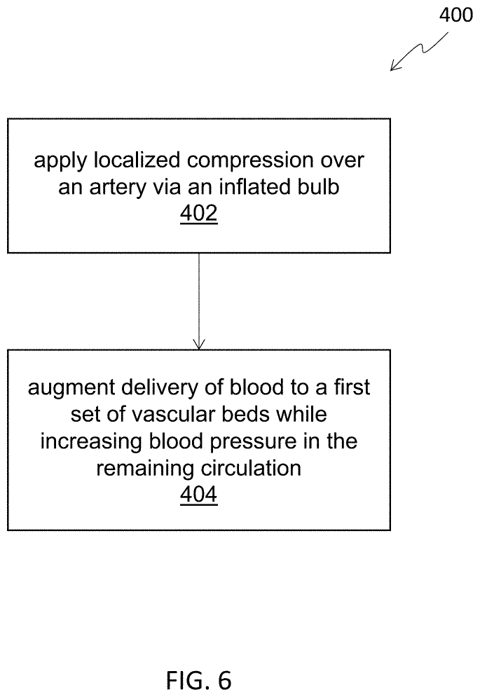

With reference now to FIG. 6, a method 400 for enhancing blood supply to vital organs is described that can be implemented with embodiment of the device described above. In one embodiment, the method 400 includes the steps of applying localized compression over an artery via an inflated bulb 402, and augmenting delivery of blood to increase blood pressure in the remaining circulation 404. In one embodiment, the step of applying localized compression includes applying compression over an artery supplying an extremity. In one embodiment, the step of applying localized compression includes applying compression comprises applying compression over two to four sets of arteries each supplying two to four separate extremities. In one embodiment, the step of applying localized compression over an artery includes filling a bulb with air until a threshold pressure is detected. In one embodiment, the step of applying localized compression over an artery includes filling a bulb with air until a threshold physiological parameter is detected. In one embodiment, the step of applying localized compression over an artery includes filling a bulb with air until a threshold flow occlusion parameter is detected and then maintaining that filled bulb at the occlusion parameter. In one embodiment, the step of applying localized compression over an artery via an inflated bulb includes strapping the bulb to a surface patent's skin over the artery. In one embodiment, the bulb will maintain inflation to occlusion parameter until a safety feature time of one hour is reached, at which point it will automatically deflate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A vascular compression device comprising:
a first compression loop having a first pneumatic bulb configured to inflate and deflate;
a second compression loop having a second pneumatic bulb configured to inflate and deflate; and
a pneumatic pump system connected via tubing to the first and second pneumatic bulbs.

2. The vascular compression device of claim 1, wherein the first compression loop comprises a first strap and a first fastener, and the second compression loop comprises a second strap and a second fastener.

3. The vascular compression device of claim 1, wherein the pneumatic pump system comprises a manual pump.

4. The vascular compression device of claim 1, wherein the pneumatic pump system comprises an electric air pump.

5. The vascular compression device of claim 1, wherein the pneumatic pump system is configured to be inflated with compressed air, O² or CO².

6. The vascular compression device of claim 1 further comprising a pressure gauge connected to the tubing between the first and second pneumatic bulbs and the pneumatic pump system.

7. The vascular compression device of claim 1 wherein the tubing comprises a first branch connected to the first pneumatic bulb and a second branch connected to the second pneumatic bulb, and wherein the first and second branch merge into a third branch connected to the pneumatic pump system.

8. The vascular compression device of claim 1 further comprising:
a controller configured to communicate with the pneumatic system for inflating, holding inflation, or deflating the first and second pneumatic bulbs.

9. The vascular compression device of claim 8, wherein at least one of the first and second pneumatic bulbs comprises a pressure sensor communicatively connected to the controller.

10. The vascular compression device of claim 8, wherein at least one of the first and second pneumatic bulbs comprises a physiological sensor communicatively connected to the controller.

11. The vascular compression device of claim 8, wherein the first pneumatic bulb has a first sensor communicatively connected to the controller and configured to detect flow occlusion, and the second pneumatic bulb has a second sensor communicatively connected to the controller and configured to detect flow occlusion.

12. The vascular compression device of claim 8, wherein the controller is configured to generate signals for inflating or deflating the first and second pneumatic bulbs based on feedback from first and second pressure sensors.

13. The vascular compression device of claim 8, wherein the controller is configured to generate a sound cue based on feedback from first and second pressure sensors.

14. The vascular compression device of claim 1, at least one end of the of the firsts and second compression loops has a cuff slide portion comprising a firm, low friction surface.

15. The vascular compression device of claim 1, wherein the tubing is configured to detach from the first and second pneumatic bulbs.

16. A method for enhancing blood supply to vital organs comprising:
applying localized external compression over an artery via an inflated bulb, and
augmenting delivery of blood to increase blood pressure in the remaining circulation.

17. The method of claim 16, wherein the step of applying localized compression further comprises applying compression over an artery supplying any or all extremities.

18. The method of claim 16, wherein the step of applying localized compression further comprises applying compression comprises applying compression over two to four sets of arteries each supplying two to four separate extremities.

19. The method of claim 16, wherein the step of applying localized compression further comprises applying compression over three or four sets of arteries each supplying three or four separate extremities.

20. The method of claim 16, wherein the step of applying localized compression over an artery further comprises filling a bulb with air until a threshold pressure is detected and then maintaining that pressure.

21. The method of claim 16, wherein the step of applying localized compression over an artery further comprises filling a bulb with air until a threshold physiological parameter is detected.

22. The method of claim 16, wherein the step of applying localized compression over an artery further comprises filling a bulb with air until a threshold flow occlusion parameter is detected.

23. The method of claim 16, wherein the step of applying localized compression over an artery via an inflated bulb further comprises strapping the bulb to a surface patent's skin over the artery.
